(19) United States
(12) Patent Application Publication
(10) Pub. No.: US 2022/0041550 A1
(43) Pub. Date: Feb. 10, 2022

(54) USE OF AT LEAST ONE HEMI-PEROXYACETAL, ALONE OR IN COMBINATION WITH OTHER PEROXIDES, TO PROMOTE POLYMERISATION OR COPOLYMERISATION OF ETHYLENE UNDER HIGH PRESSURE

(71) Applicant: ARKEMA FRANCE, Colombes (FR)

(72) Inventors: Bruno VAN HEMELRYCK, Pierre-Benite Cedex (FR); Vincent MENENTEAU, Pierre-Benite Cedex (FR)

(21) Appl. No.: 17/415,884

(22) PCT Filed: Dec. 18, 2019
(86) PCT No.: PCT/FR2019/053152

§ 371 Date: Jun. 18, 2021

(30) Foreign Application Priority Data
Dec. 21, 2018 (FR) 1873745

(51) Int. Cl. C07C 409/22; C08F 10/02; C08F 2/01

(52) U.S. Cl. CPC C07C 409/22; C08K 5/14; C08F 2/01; C08F 10/02

(57) ABSTRACT

The present invention relates to the use of at least one peroxide selected from the group consisting of hemiperoxyacetals, alone or in combination with one or more distinct additional peroxides, for the radical polymerization or copolymerization of ethylene under high pressure.

The invention also concerns a process for preparing polyethylene, comprising a step of radical polymerization or copolymerization of ethylene under high pressure in the presence of at least one peroxide selected from the group consisting of hemiperoxyacetals, alone or in combination with one or more distinct additional peroxides.

The present invention likewise pertains to a composition comprising ethylene, at least one peroxide selected from the group consisting of hemiperoxyacetals, and optionally one or more additional peroxides distinct from hemiperoxyacetals.

The present invention relates to the use of at least one peroxide selected from the group consisting of hemiperoxyacetals, as defined hereinafter, alone or in combination with one or more distinct additional peroxides, for the radical polymerization or copolymerization of ethylene under high pressure.

The invention also concerns a process for preparing polyethylene, especially low-density polyethylenes (LDPEs), or an ethylene copolymer, especially copolymers of ethylene and vinyl acetate (EVA), (meth)acrylic ethylene copolymers, copolymers based on ethylene and at least one alpha- or alpha, omega-olefin, copolymers based on ethylene and carbon monoxide, and copolymers based on ethylene and unsaturated cyclic anhydride comonomers, comprising a step of radical polymerization or copolymerization of ethylene under high pressure in the presence of at least one peroxide selected from the group consisting of hemiperoxyacetals, as defined hereinafter, alone or in combination with one or more distinct additional peroxides.

The present invention likewise pertains to a composition comprising ethylene, at least one peroxide selected from the group consisting of hemiperoxyacetals, as defined hereinafter, and optionally one or more additional peroxides distinct from hemiperoxyacetals.

Low-density polyethylenes (also called LDPEs) and ethylene copolymers are generally prepared in an autoclave or tubular reactor under high pressure, by continuous introduction of ethylene, of one or more optional comonomers and of one or more initiators, such as organic peroxides, which are usually diluted in an organic solvent. The pressure inside the reactor is generally between 500 and 5000 bar, whereas the temperature at initiation of the reaction varies usually from 80 to 250° C. The maximum reaction temperature is typically between 120 and 350° C. The polymerization initiators may be injected into one or more reaction zones of the reactor.

The search for gains in production, and consequently for cost rationalization, while retaining high reliability of the process carried out, is a constant preoccupation for producers of polyethylene and ethylene copolymers. In particular, it is important to develop a process which allows the manufacture of polyethylene or of ethylene copolymers that is of high productivity, accompanied by appropriate rationalization of the production costs, while retaining high reliability.

These searches for improvement in the implementation of this type of process are even more important given the commercial interest in polyethylenes and ethylene copolymers, since these polymers can be used in diverse fields of application, owing to their compatibility with many other polymers or resins.

Thus, low-density polyethylenes are commonly used in the manufacture of films within the sector of packaging or flexible bottling.

Moreover, ethylene copolymers can be used in the manufacture of cables, of hotmelt adhesive compositions, of multilayer packaging films or of masterbatches. They may also be used as an impact modifier in the production of polymers such as polyamides and polyesters for the electronic and automotive sectors.

The organic peroxides that are conventionally used may be compounds having the capacity to initiate a radical polymerization or copolymerization reaction of ethylene under high pressure within a temperature range—referred to as the middle temperature range—of from 160° C. to 190° C. These organic peroxides are, especially, peroxy esters, more particularly tert-butyl peroxy-2-ethylhexanoate, sold under the trade name Luperox® 26, and tert-amyl peroxy-2-ethylhexanoate, sold under the trade name Trigonox® 121.

For example, patent application US 2006/0149004 describes a continuous preparation method for polyethylene comprising a step of radical polymerization of ethylene under high pressure in the presence of peroxides that are capable of initiating the radical reaction within a temperature range of from 160° to 190° C., including tert-butyl peroxy-2-ethylhexanoate, tert-butylperoxy acetate and tert-butylperoxy benzoate.

The organic peroxides conventionally used may also be compounds which are capable of initiating a radical polymerization or copolymerization reaction of ethylene under high pressure within a higher temperature range, referred to as high-temperature range, of from 190° C. to 250° C. These organic peroxides are, especially, peroxy esters, such as tert-butyl peroxy-3,5,5-trimethylhexanoate, sold under the trade name Luperox® 270, tert-butyl peracetate, sold under the trade name Luperox® 7, or else tert-butyl perbenzoate, sold under the trade name Luperox® P.

Thus, in the implementation of a process for preparing polyethylene or an ethylene copolymer, it is conventional practice to inject a peroxy ester or a collective of peroxy esters (commonly referred to as a cocktail of peroxy esters) into one or more reaction zones of a reactor in order to bring about the radical polymerization or copolymerization of ethylene under high pressure. More particularly, it is known practice to inject a cocktail of peroxy esters which are capable of initiating the radical polymerization or copolymerization reaction of ethylene within different temperature ranges, for example in a range of temperatures of from 160° C. to 190° C. and in a range of temperatures of from 190° C. to 250° C., into a number of reaction zones of a reactor. This type of system of peroxidic initiators including one initiator more reactive than the others has the advantage of covering a wide range of initiation temperatures for the ethylene polymerization or copolymerization reaction.

Organic peroxides, especially reactive peroxy esters capable of initiating a radical reaction in a temperature range from 160° C. to 190° C., however, have the drawback of possessing, generally speaking, a self-accelerating exothermic decomposition temperature which is close to the ambient temperature, especially to 35° C., which means that they have to be stored in a cold environment, typically at temperatures of around 5 to 10° C. Storage conditions of this kind are employed especially for preventing these peroxides from undergoing self-accelerating exothermic decomposition and ensuring that there is no risk of them igniting and/or exploding violently. Hence the use of these organic peroxides, alone or in the form of a cocktail, causes difficulties, in terms of storage and of security measures to be adopted during their handling and/or their transportation, and this is likely to have an adverse impact on the production costs for polyethylene and ethylene copolymers. Alternatively expressed, these peroxy esters necessitate additional storage, transport and handling operations, which are often expensive and complicated to implement, owing to their self-accelerating exothermic decomposition temperatures of typically less than 50° C.

Furthermore, these peroxy esters, used alone or as a mixture, also have the disadvantage of being consumed at substantial levels during the process for preparing polyethylene and an ethylene copolymer. More precisely, the specific peroxide consumption, defined as the amount of peroxide(s) needed to obtain a given amount of polymer, expressed typically in kilograms of peroxides per metric ton of polymer produced, still remains too high with the use of the prior-art peroxy esters.

The effects of this high peroxide consumption are not only to increase the production costs of the polyethylene and the ethylene copolymer, but also to increase the risks of giving rise to substantial amounts of volatile organic compounds resulting from the decomposition of the peroxide or peroxides employed.

The reason is that the use of additional amounts of peroxide in order to complete the radical reaction of the ethylene exacerbates the risks of decomposition of these peroxides into volatile organic compounds.

The abovementioned disadvantages therefore run counter to rationalizing the costs of an industrial-scale preparation process for polyethylene and ethylene-based copolymers, and cause environmental problems associated with the formation of volatile organic compounds, and also problems in terms of storage and transportation and/or handling difficulties which are linked to the self-accelerating exothermic decomposition temperatures of the highly reactive peroxy esters commonly employed in the prior art.

One of the objectives of the present invention, therefore, is to propose a compound or an association of compounds that is capable of initiating the radical polymerization or copolymerization reaction of ethylene under high pressure in a reliable way while reducing the amount of initiator(s) consumed during the reaction and be easier to store relative to the peroxy esters conventionally used in the prior art.

In other words, there is a genuine need for effective substitution of some or all of the peroxy esters conventionally used in the radical polymerization or copolymerization of ethylene under high pressure with a system containing one or more initiators that is easy to store, handle and/or transport, and which is capable of reducing the specific consumption of initiator(s) employed in the radical polymerization or copolymerization reaction.

More particularly, one of the objectives of the present invention is to provide a system containing one or more initiators for the radical polymerization or copolymerization of ethylene under high pressure so as to enhance the productivity and the rationalization of the production costs of a process for preparing polyethylene or an ethylene-based copolymer.

A particular subject of the present invention is the use of at least one organic peroxide, alone or in combination with one or more distinct additional organic peroxides, selected from the group consisting of hemiperoxyacetals, preferably having a half-life temperature at one minute and at atmospheric pressure of from 125° C. to 160° C., for the radical polymerization or copolymerization of ethylene under high pressure.

The organic peroxide according to the invention is used in particular, alone or in combination with one or more distinct additional organic peroxides, for initiating the polymerization or copolymerization of ethylene by a radical route under high pressure.

In other words, the invention proposes using a peroxidic initiator system composed of one or more organic peroxides in which at least one organic peroxide is selected from the group consisting of hemiperoxyacetals, preferably having a half-life temperature at one minute and at atmospheric pressure of from 125° C. to 160° C., for the radical polymerization or copolymerization of ethylene under high pressure, especially for initiating the radical polymerization or copolymerization of ethylene under high pressure.

An advantage of the organic peroxide as defined above, alone or in combination with one or more distinct additional organic peroxides, is the effective initiation of the radical polymerization or copolymerization of ethylene under high pressure so as to produce polyethylene or an ethylene-based copolymer.

Accordingly, the use of the organic peroxide according to the invention enables effective replacement of some or all of the peroxy esters conventionally used in the prior art for the radical polymerization or copolymerization of ethylene under high pressure.

Furthermore, another advantage of the organic peroxide according to the invention is that it has a self-accelerating exothermic decomposition temperature of more than 50° C. and so can be stored, transported and handled at ambient temperature, and hence under conditions that are easier to implement than the peroxy esters conventionally used in the prior art, which must generally be stored in a cold environment at temperatures in the region of 5 to 10° C. This means that the use of the organic peroxide according to the invention is less restrictive than that of the peroxy esters conventionally employed in the radical polymerization or copolymerization of ethylene.

Expressed alternatively, the costs associated with the storage, transport and handling of the organic peroxide according to the invention are reduced relative to the costs engendered by the use of the peroxy esters utilized in the prior art. Similarly, the use of the organic peroxide according to the invention enhances the safety and reliability of a process for preparing polyethylene or ethylene-based copolymer(s).

Moreover, the peroxidic initiator system according to the invention enables a reduction in the specific consumption of initiator(s) for a given injection zone, whether the consumption of the organic peroxide, as defined above, when it is used alone, or the overall consumption of the mixture of organic peroxides containing the organic peroxide as defined above and the additional organic peroxide(s).

More generally, the specific consumption of the organic peroxide according to the invention, when it is used alone, and the specific overall consumption of a combination of organic peroxides based on the organic peroxide according to the invention and on one or more additional organic peroxides, are reduced during the radical polymerization or copolymerization of ethylene under high pressure, relative to the peroxy esters used in the prior art.

Consequently, the organic peroxide according to the invention or the system containing an organic peroxide of this kind has an economic advantage associated with the limitation to the costs of producing polyethylene or ethylene-based copolymer(s), and an environmental advantage linked to the limitation to risks of producing volatile organic compounds resulting from the breakdown of the organic peroxide(s) employed.

In the sense of the present invention, “specific consumption of initiator(s)” refers to the amount in kilograms of initiator that is needed to produce one metric ton of polymer (resin). The specific consumption of initiator(s) may also be expressed in grams of initiator(s) per kilogram of polymer obtained.

Moreover, the peroxidic initiator system according to the invention is able to span a wide range of initiation temperatures for the radical polymerization or copolymerization of ethylene.

The invention also concerns a process for preparing polyethylene or an ethylene copolymer, comprising a step of radical polymerization or copolymerization of ethylene under high pressure in the presence of at least one organic peroxide selected from the group consisting of hemiperoxyacetals, preferably having a half-life temperature at one minute and at atmospheric pressure of from 125° C. to 160° C., alone or in combination with one or more distinct additional organic peroxides.

The preparation process according to the invention exhibits a high productivity and an improved reliability.

The reason is that the costs associated with the storage, transport and handling of the organic peroxide according to the invention are reduced relative to the costs engendered by the use of peroxy esters.

The present invention also pertains to a composition comprising:
(i) at least one ethylene monomer,
(ii) at least one peroxide selected from the group consisting of hemiperoxyacetals, preferably having a half-life temperature at one minute and at atmospheric pressure of from 125° C. to 160° C., and
(iii) optionally at least one additional organic peroxide distinct from the peroxide (ii).

The composition enables a reduction in the specific consumption of initiator(s) relative to the peroxy esters conventionally used in the preparation of polyethylene or of ethylene copolymer(s).

The composition according to the invention allows the polymerization to result in polyethylene or an ethylene-based copolymer.

The composition according to the invention is therefore polymerizable or able to polymerize.

In the sense of the present invention, “high pressure” means a pressure greater than 50 MPa. Preferably, the pressure varies from 500 bar (50 MPa) to 3000 bar (300 MPa), preferentially from 1200 bar (120 MPa) to 3000 bar (300 MPa).

Other features and advantages of the invention will emerge more clearly on reading the description and the examples that follow.

In the following text, and unless indicated otherwise, the limits of a range of values are included in said range.

The expression “at least one” is equivalent to the expression “one or more”.

Peroxidic Initiator System

As indicated above, the present invention proposes the use of a peroxidic initiator system, namely a system containing at least one organic peroxide, as defined above, alone or in combination with one or more distinct additional organic peroxides.

The peroxidic initiator system may be either the organic peroxide as defined above, used alone, or a combination of the organic peroxide and one or more distinct additional organic peroxides as defined hereinafter.

With the peroxidic initiator system it is possible to initiate the radical polymerization or copolymerization of ethylene under high pressure.

In the sense of the present invention, “combination” means that the at least one organic peroxide, as defined above, is in a mixture with one or more distinct additional organic peroxides in a single formulation, or that said at least one organic peroxide and the additional organic peroxide or peroxides are in distinct formulations.

In other words, “combination” means that the at least one organic peroxide as defined above and the additional organic peroxide or peroxides belong to a single overall system of initiators which is capable of effecting the polymerization or copolymerization of ethylene by a radical route under high pressure.

This means that the at least one organic peroxide as defined above and the distinct additional organic peroxide or peroxides participate overall in the polymerization or copolymerization of ethylene by a radical route under high pressure, depending on the temperature at which they initiate the reaction, and that they are not necessarily formulated in the same composition.

Thus, for example, the organic peroxide as defined above may be injected at one point of a reactor, while the additional organic peroxide or peroxides may be injected at another point of the reactor.

According to one embodiment, the organic peroxide as defined above is in a mixture with one or more distinct additional organic peroxide(s), i.e. they are formulated in the same composition. In this case, the combination corresponds to a mixture of the at least one organic peroxide according to the invention and one or more additional organic peroxide(s).

Organic Peroxide (Hemiperoxyacetal)

The at least one organic peroxide used in accordance with the present invention is selected from the group consisting of hemiperoxyacetals.

The term “hemiperoxyacetal” means a compound of the general formula (R₃)(R₄)C(—OR₁)(—OOR₂), in which:
R₁ represents a linear or branched, preferably C₁-C₁₂, preferably C₁-C₄, and more preferably C₁, alkyl group, or represents a cycloalkyl group with R₂,
R₂ represents a linear or branched, preferably C₁-C₁₂, preferably C₄-C₁₂, and more preferably C₅, alkyl group, or represents a cycloalkyl group with R₁,
R₃ represents a hydrogen atom or a linear or branched, preferably C₁-C₁₂, more preferably C₄-C₁₂, alkyl group, or represents a cycloalkyl group with R₄,
R₄ represents a hydrogen atom or a linear or branched, preferably C₁-C₁₂, more preferably C₄-C₁₂, alkyl group, or represents a cycloalkyl group with R₃.

Preferably, R₃ forms a cycloalkyl group with R₄.

Preferably, when R₃ is a hydrogen atom, R₄ is a linear or branched, preferably C₁-C₁₂, more preferably C₄-C₁₂, alkyl group.

The organic peroxide is preferably selected from the group consisting of hemiperoxyacetals having a half-life temperature at one minute and at atmospheric pressure of from 125° C. to 160° C., preferably from 130° C. to 155° C. and more preferably from 140° C. to 150° C.

The term “half-life temperature at one minute” represents the temperature at which half of the organic peroxide has decomposed in a one minute and at atmospheric pressure. Conventionally, the “half-life temperature at one minute” is measured in n-decane or n-dodecane.

The organic peroxide used according to the invention advantageously enables the radical reaction of ethylene, or of ethylene with any monomer copolymerizable with ethylene, by a radical route to be initiated under high pressure in a range of temperatures, referred to as medium temperature range, which may be from 160° C. to 190° C.

Accordingly, the use of the organic peroxide according to the invention enables an effective replacement for the conventional peroxy esters initiating the radical polymerization or copolymerization of ethylene within a range of temperatures, referred to as medium temperature range, which may be from 160 to 190° C.

The organic peroxide according to the invention is preferably selected from the group consisting of hemiperoxyacetals conforming to the general formula (I) below:

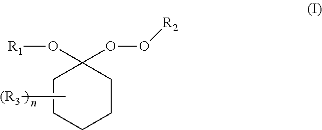

in which formula (I):
R₁ represents a linear or branched C₁-C₄, preferably C₁, alkyl group,
R₂ represents a branched C₄-C₁₂, preferably C₅, alkyl group,
n denotes zero or an integer from 1 to 3,
R₃ represents a linear or branched C₁-C₃ alkyl group.

R₁ preferably represents a linear, more particularly C₁-C₂, more preferably C₁, alkyl group.

R₂ preferably represents a branched C₄-C₅, more preferably C₅, alkyl group.

Preferably n denotes zero.

R₃ preferably represents a linear or branched, C₁-C₂, more preferably C₁, alkyl group. Preferably, in the formula (I), R₁ represents a linear or branched, C₁-C₂ alkyl group, R₂ represents a branched C₄-C₅ alkyl group, and n denotes zero.

More preferably still, in the formula (I), R₁ represents a C₁ alkyl group, R₂ represents a branched C₅ alkyl group and n denotes zero.

The organic peroxide or peroxides is or are preferably selected from the group consisting of 1-methoxy-1-tert-amylperoxycyclohexane (TAPMC), 1-methoxy-1-t-butylperoxycyclohexane (TBPMC), 1-methoxy-1-t-amylperoxy-3,3,5-trimethylcyclohexane, 1-methoxy-1-t-butylperoxy-3,3,5-trimethylcyclohexane, 1-ethoxy-1-t-amylperoxycyclohexane, 1-ethoxy-1-t-butylperoxycyclohexane, 1-ethoxy-1-t-butyl-3,3,5-peroxycyclohexane and mixtures thereof.

More preferably still, the organic peroxide according to the invention is 1-methoxy-1-tert-amylperoxycyclohexane (TAPMC).

Accordingly, the invention concerns the use of 1-methoxy-1-tert-amylperoxycyclohexane, formulated alone or in combination with one or more distinct organic peroxides, for the radical polymerization or copolymerization of ethylene under high pressure, preferably the radical polymerization of ethylene under high pressure.

Distinct Additional Organic Peroxide(s)

According to one embodiment, the at least one organic peroxide according to the invention is used in combination with one or more distinct additional organic peroxides, so as to cover a more extended temperature range for initiation of the radical polymerization or copolymerization of ethylene under high pressure than the range obtained with the organic peroxide according to the invention and used on its own.

By “distinct additional organic peroxide” is meant that the additional organic peroxide or peroxides is or are structurally distinct from the organic peroxide according to the invention and selected from the group of the hemiperoxyacetals as defined above.

The additional organic peroxide or peroxides is or are selected preferably from the group consisting of peroxyacetals capable of initiating the radical polymerization or copolymerization of ethylene under high pressure within a temperature range of from 190° C. to 250° C.

The peroxidic initiation system may therefore comprise one or more organic peroxides as described above, enabling the initiation of the radical reaction of ethylene under high pressure within a temperature range, referred to as the medium temperature range, of from 160° C. to 190° C., and one or more additional organic peroxides selected from the group consisting of peroxyacetals and capable of initiating the radical reaction of ethylene under high pressure within a temperature range, referred to as high temperature range, of from 190° C. to 250° C.

In other words, in accordance with this embodiment, the peroxidic initiator system covers a range of temperatures for initiation of the reaction of ethylene by a radical route under high pressure, of from 160° C. to 250° C.

The additional organic peroxide or peroxides is or are preferably selected from the group consisting of peroxyacetals, preferably having a half-life temperature at one minute and at atmospheric pressure of from 145 to 180° C., preferably from 150° C. to 180° C., more preferably from 160° C. to 175° C., and very preferably from 160° C. to 170° C.

The additional organic peroxide or peroxides is or are preferably selected from the group consisting of the peroxyacetals conforming to the general formula (II) below:

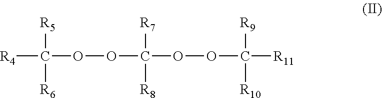

in which formula (II) R₄ to R₁₁, which are identical or different, represent a linear or branched C₁-C₆ alkyl group.

R₄ to R₁₁, which are identical or different, preferably represent a linear C₁-C₆ group, more preferably methyl, ethyl, n-propyl, n-butyl, n-pentyl or n-hexyl.

Preferably, in the formula (II), R₅, R₆, R₉ and R₁₀, which are identical or different, represent a linear or branched C₁-C₅, more preferably C₁, alkyl group.

Preferably, in the formula (II), R₇ represents a linear or branched C₁-C₄, more preferably C₁, alkyl group.

Preferably, in the formula (II), R₄ and R₁₁, which are identical or different, represent a linear or branched C₂-C₄, more preferably C₁, alkyl group.

Preferably, in the formula (II), R₇ and R₈, which are identical or different, represent a linear C₁-C₆ alkyl group.

Preferably, in the formula (II):
R₅, R₆, R₉ and R₁₀ represent a C₁ alkyl group,
R₄ and R₁₁, which are identical or different, represent a linear or branched C₂-C₄, preferably C₂, alkyl group,
R₇ and R₈, which are identical or different, represent a linear C₁-C₆, preferably, identically or differently, C₁-C₄, alkyl group, and more preferably R₇ and R₈ are different and represent a linear C₁-C₂ alkyl group.

The additional organic peroxide or peroxides is or are preferably selected from the group consisting of 2,2-di(tert-amylperoxy)propane, 2,2-di(tert-amylperoxy)butane and mixtures thereof.

Even more preferentially, the additional organic peroxide is 2,2-di(tert-amylperoxy)butane, which is sold under the trade name Luperox® 520.

As a variant, the additional organic peroxide(s) distinct from the organic peroxide, as described above, may be a mixture based on 2,2-di(tert-amylperoxy)propane and 2,2-di(tert-amylperoxy)butane.

The invention advantageously concerns the use of 1-methoxy-1-tert-amylperoxycyclohexane in combination with one or more additional organic peroxides selected from the group consisting of 2,2-di(tert-amylperoxy)propane, 2,2-di(tert-amylperoxy)butane and mixtures thereof for the radical polymerization or copolymerization of ethylene under high pressure.

More preferably, the invention concerns the use of 1-methoxy-1-tert-amylperoxycyclohexane in combination with 2,2-di(tert-amylperoxy)butane for the radical polymerization or copolymerization of ethylene under high pressure, preferably for the polymerization of ethylene.

More particularly, the combination of at least one organic peroxide according to the invention and one or more additional organic peroxides selected from the group consisting of peroxyacetals, as defined above, leads to good results in terms of a gain in the overall specific consumption of the initiators engaged in the radical reaction of ethylene, especially in relation to the combination of tert-butyl peroxy-2-ethylhexanoate (Luperox® 26) and tert-butyl peroxy-3,5,5-trimethylhexanoate (Luperox® 270).

Alternatively, the additional organic peroxide or peroxides is or are selected from the group consisting of cyclic peroxyacetals, preferably having a half-life temperature at one minute and at atmospheric pressure of from 145 to 180° C., more preferably from 145° C. to 170° C., more preferably still from 150° C. to 160° C.

The cyclic peroxyacetal or peroxyacetals are preferably selected from the group consisting of 1,1-di(tert-amylperoxy)cyclohexane (Luperox® 531), 1,1-di(tert-butylperoxy)-3,3,5-trimethylcyclohexane (Luperox® 231), and 1,1-di(tert-butylperoxy)cyclohexane (Luperox® 331), preference being given to 1,1-di(tert-amylperoxy)cyclohexane.

According to another embodiment, the additional organic peroxide or peroxides may also be selected from the group consisting of peroxides having a half-life temperature, at one minute and at atmospheric pressure, which is lower than the half-life temperature of the hemiperoxyacetal as defined above, or, in the case of several hemiperoxyacetals as defined above, of the hemiperoxyacetal having the lowest half-life temperature at one minute and at atmospheric pressure among the hemiperoxyacetals.

The additional organic peroxide or peroxides is or are preferably therefore selected from the group consisting of tert-amyl peroxyneodecanoate, sold under the trade name Luperox® 546, tert-butyl peroxyneodecanoate, sold under the trade name Luperox® 10, diethylhexyl peroxydicarbonate, sold under the trade name Luperox® 223, tert-amyl perpivalate, sold under the trade name Luperox® 554, tert-butyl perpivalate, sold under the trade name Luperox® 11, di(3,5,5-trimethylhexanoyl) peroxide, sold under the trade name Luperox® 219, 2,5-dimethyle-2,5-di(2-ethylhexanoylperoxy)hexane, sold under the trade name Luperox® 256, and tert-amyl peroxy-2-ethylhexanoate, sold under the trade name Luperox® 575.

According to one advantageous embodiment, the peroxidic initiator system comprises:
one or more organic peroxides selected from the group consisting of hemiperoxyacetals as defined above,
one or more additional organic peroxides selected from the group consisting of peroxyacetals as defined above,
one or more additional organic peroxides having a half-life temperature, at one minute and at atmospheric pressure, which is lower than the half-life temperature of the hemiperoxyacetal as defined above, or, in the case of several hemiperoxyacetals as defined above, of the hemiperoxyacetal having the lowest half-life temperature at one minute and at atmospheric pressure among the hemiperoxyacetals.

The peroxidic initiator system preferably comprises 1-methoxy-1-tert-amylperoxycyclohexane (TAPMC), 2,2-di(tert-amylperoxy)butane (Luperox® 520) and tert-butyl perpivalate (Luperox® 11).

The reason is that the cocktail of initiators composed of 1-methoxy-1-tert-amylperoxycyclohexane, 2,2-di(tert-amylperoxy)butane and tert-butyl perpivalate leads to good results in terms of a gain on the overall specific consumption of the initiators engaged in the radical reaction of ethylene.

According to another embodiment, the additional organic peroxide or peroxides may also be selected from the group consisting of peroxides having a half-life temperature, at one minute and at atmospheric pressure, which is higher than the half-life temperature of the peroxide selected from the group consisting of hemiperoxyacetals as defined above.

According to this embodiment, the additional organic peroxide or peroxides is or are different from the peroxyacetals described above.

The additional organic peroxide or peroxides is or are preferably selected from the group consisting of tert-amyl peroxy-3,5,5-trimethylehexanoate, sold under the trade name Luperox® 570, tert-butyl peroxy-3,5,5-trimethylhexanoate, sold under the trade name Luperox® 270, tert-butyl peracetate, sold under the trade name Luperox® 7, 2,2-di(tert-amylperoxy)butane, sold under the trade name Luperox® 520, 2,2-di(tert-butylperoxy)butane, sold under the trade name Luperox® 220, tert-butyl peroxybenzoate, sold under the trade name Luperox® P, n-butyl 4,4-di(tert-butylperoxy)valerate, sold under the trade name Luperox® 230, ethyl 3,3-di(tert-amylperoxy)butyrate, sold under the trade name Luperox® 533, and mixtures thereof.

According to one advantageous embodiment, the peroxidic initiator system comprises:
one or more organic peroxides selected from the group consisting of hemiperoxyacetals as defined above,
one or more additional organic peroxides selected from the group consisting of peroxides having a half-life temperature at one minute and at atmospheric pressure which is less than the half-life temperature of said at least one hemiperoxyacetal as defined above,
one or more additional organic peroxides selected from the group consisting of peroxides having a half-life temperature at one minute and at atmospheric pressure which is higher than the half-life temperature of the peroxide selected from the group consisting of hemiperoxyacetals as defined above.

The peroxidic initiator system preferably comprises 1-methoxy-1-tert-amylperoxycyclohexane (TAPMC), tert-butyl perpivalate (Luperox® 11) and tert-butyl peroxy-3,5,5-trimethylhexanoate (Luperox® 270).

More preferably, the peroxidic initiator system is a composition comprising 1-methoxy-1-tert-amyl peroxycyclohexane, tert-butyl perpivalate and tert-butyl peroxy-3,5,5-trimethylhexanoate. Expressed alternatively, 1-methoxy-1-tert-amyl peroxycyclohexane, tert-butyl perpivalate and tert-butyl peroxy-3,5,5-trimethylhexanoate are formulated in the same composition.

According to one advantageous embodiment, the peroxidic initiator system comprises:
one or more organic peroxides selected from the group consisting of hemiperoxyacetals as defined above,
one or more additional organic peroxides selected from the group consisting of peroxyacetals as defined above,
one or more additional organic peroxides selected from the group of peroxides having a half-life temperature, at one minute and at atmospheric pressure, which is higher than the half-life temperature of the hemiperoxyacetal as defined above, or, in the case of several hemiperoxyacetals as defined above, of the hemiperoxyacetal having the highest half-life temperature at one minute and at atmospheric pressure among the hemiperoxyacetals.

More preferably, the peroxidic initiator system comprises 1-methoxy-1-tert-amylperoxycyclohexane (TAPMC), 2,2-di(tert-amylperoxy)butane (Luperox® 520) and tert-butyl peroxypivalate (Luperox® 11).

More preferably still, the peroxidic initiator system comprises 1-methoxy-1-tert-amylperoxycyclohexane, 2,2-di(tert-amylperoxy)propane, 2,2-di(tert-amylperoxy)butane (Luperox® 520) and tert-butyl peroxypivalate (Luperox® 11).

According to another embodiment, the additional organic peroxide or peroxides may also be selected from the group consisting of peroxy esters which are capable of initiating the radical polymerization or copolymerization of ethylene under high pressure at a temperature of from 160° C. to 190° C.

The peroxy esters are preferably selected from the group consisting of tert-butyl peroxy-2-ethylhexanoate (Luperox® 26), tert-amyl peroxy-2-ethylhexanoate, sold under the trade name Trigonox® 121, and mixtures thereof.

More preferably the peroxy ester is tert-butyl peroxy-2-ethylhexanoate.

According to another embodiment, the additional organic peroxide or peroxides may also be selected from the group consisting of organic peroxides which are capable of initiating the radical polymerization or copolymerization of ethylene under high pressure at a temperature of more than 220° C.

The additional organic peroxide or peroxides are preferably selected from the group consisting of di-tert-amyl peroxide, sold under the trade name Luperox® DTA, 2,5-dimethyl-2,5-di(tert-butylperoxy)hexane, sold under the trade name Luperox® 101, di-tert-butyl peroxide, sold under the trade name Luperox® DI, 3,6,9-triethyl-3,6,9-trimethyl-1,4,7-triperoxonane, sold under the trade name Trigonox® 301, or else 3,3,5,7,7-15 pentamethyl-1,2,4-trioxepane, sold under the trade name Trigonox® 311, and mixtures thereof.

According to one advantageous embodiment, the peroxidic initiator system comprises:
one or more organic peroxides selected from the group consisting of hemiperoxyacetals as defined above,
one or more additional organic peroxides selected from the group consisting of peroxyacetals as defined above,
one or more additional organic peroxides selected from the group consisting of organic peroxides capable of initiating the radical polymerization or copolymerization of ethylene under high pressure at a temperature of more than 220° C.

The peroxidic initiator system preferably comprises 1-methoxy-1-tert-amylperoxycyclohexane (TAPMC), 2,2-di(tert-amylperoxy)butane (Luperox® 520) and di-tert-butyl peroxide (Luperox® DI).

The reason is that the cocktail of initiators composed of 1-methoxy-1-tert-amylperoxycyclohexane, 2,2-di(tert-amylperoxy)butane and di-tert-butyl peroxide leads to good results in terms of a gain on the overall specific consumption of the initiators engaged in the radical reaction of ethylene.

The peroxidic initiator system advantageously comprises:
one or more organic peroxides selected from the group consisting of hemiperoxyacetals as defined above,
one or more additional organic peroxides selected from the group consisting of peroxyacetals as defined above,
one or more additional organic peroxides selected from the group consisting of peroxides having a half-life temperature, at one minute and at atmospheric pressure, which is lower than the half-life temperature of the hemiperoxyacetal as defined above, or, in the case of several hemiperoxyacetals as defined above, of the hemiperoxyacetal having the lowest half-life temperature at one minute and at atmospheric pressure among the hemiperoxyacetals,
one or more additional organic peroxides selected from the group consisting of organic peroxides capable of initiating the radical polymerization or copolymerization of ethylene under high pressure at a temperature of more than 220° C.

The peroxidic initiator system preferably comprises 1-methoxy-1-tert-amylperoxycyclohexane (TAPMC), 2,2-di(tert-amylperoxy)butane (Luperox® 520), di-tert-butyl peroxide (Luperox® DI) and tert-butyl perpivalate (Luperox® 11).

More preferably the peroxidic initiator system comprises 1-methoxy-1-tert-amylperoxycyclohexane (TAPMC), 2,2-di(tert-amylperoxy)butane (Luperox® 520), 2,2-di(tert-amylperoxy)propane, di-tert-butyl peroxide (Luperox® DI) and tert-butyl perpivalate (Luperox® 11).

According to one advantageous embodiment, the peroxidic initiator system comprises:
one or more organic peroxides selected from the group consisting of hemiperoxyacetals as defined above,
one or more additional organic peroxides selected from the group consisting of peroxyacetals as defined above, and
optionally one or more additional organic peroxides selected:
a) from the group consisting of peroxides having a half-life temperature at one minute and at atmospheric pressure which is less than the half-life temperature of hemiperoxyacetals as defined above or, in the case of several hemiperoxyacetals as defined above, of the hemiperoxyacetal having the lowest half-life temperature at one minute and at atmospheric pressure among the hemiperoxyacetals,
b) from the group consisting of peroxides having a half-life temperature at one minute and at atmospheric pressure which is higher than the half-life temperature of the organic peroxide selected from the group consisting of hemiperoxyacetals as defined above,
c) from the group consisting of peroxy esters capable of initiating the radical polymerization or copolymerization of ethylene under high pressure at a temperature of from 160° C. to 190° C.,
d) from the group consisting of organic peroxides capable of initiating the radical polymerization or copolymerization of ethylene under high pressure at a temperature of more than 220° C.,
e) and mixtures thereof.

According to this embodiment, the peroxidic initiator system is a mixture; i.e., the organic peroxides are present in the same formulation.

Use

As indicated above, the organic peroxide according to the invention is used, alone or in combination with one or more additional organic peroxides, for preparing polyethylene or an ethylene copolymer, preferably polyethylene.

The organic peroxide according to the invention is preferably used, alone or in combination with one or more additional organic peroxides, for the radical polymerization of ethylene under high pressure.

Process

The present invention also concerns a process for preparing polyethylene or an ethylene copolymer, comprising a step of radical polymerization or copolymerization of ethylene under high pressure in the presence of at least one organic peroxide selected from the group consisting of hemiperoxyacetals, as described above, alone or in combination with one or more distinct additional organic peroxides as defined above.

The organic peroxide selected from the group consisting of hemiperoxyacetals is as defined above.

The organic peroxide is preferably selected from the group consisting of the hemiperoxyacetals conforming to the formula (I), and more preferably is 1-methoxy-1-tert-amylperoxycyclohexane.

The additional organic peroxide or peroxides is or are as defined above.

The additional organic peroxide or peroxides is or are preferably selected from the group consisting of peroxyacetals conforming to the formula (II), more preferably from the group consisting of 2,2-di(tert-amylperoxy)propane, 2,2-di(tert-amylperoxy)butane and mixtures thereof.

The organic peroxide according to the invention, alone or in combination with the additional organic peroxide or peroxides, is or are generally present in a quantity by mass of from 20 to 1000 ppm, relative to the quantity by mass of ethylene.

Moreover, the organic peroxide according to the invention and the additional organic peroxide or peroxides are preferably diluted in a solvent or a mixture of solvents. The solvent or solvents may be selected from the group consisting of C₆-C₂₀ alkanes and C₄-C₁₂ alpha-olefins. The solvent or solvents is or are preferably selected from the group consisting of C₆-C₂₀, especially C₈-C₁₄, preferably C₁₂ alkanes.

The polymerization or copolymerization by a radical route may be performed in an autoclave or tubular reactor.

The reaction temperature is generally between 140° C. and 350° C.

As explained above, the radical polymerization or copolymerization of ethylene is initiated at a temperature which may be from 160° C. to 250° C., preferably from 160° C. to 190° C.

The radical polymerization or copolymerization of ethylene proceeds at a pressure of 500 bar (50 MPa) to 3000 bar (300 MPa) and preferably of 1200 bar (120 MPa) to 3000 bar (300 MPa).

The organic peroxide according to the invention, alone or in combination with one or more additional organic peroxides, may be injected at one point or at several points of a reactor. The peroxidic initiator system according to the invention may therefore be injected once or a number of times into a reactor.

When the peroxidic initiator system according to the invention is injected at several points of a reactor, the reactor is referred to as a multizone reactor, preferably a multizone autoclave reactor or a multizone tubular reactor.

More particularly, in the case of a combination injected at several points of the reactor, it will be possible to use the organic peroxide according to the invention in increasing concentrations in the various formulations. In this way, the organic peroxide according to the invention may be formulated in a plurality of formulations based on additional organic peroxide(s) as defined above, at different concentrations, so as to optimize the conversion of ethylene in the reactor. These various formulations may be injected into several points of a reactor.

Moreover, in the case of injection at several points of the reactor of an organic peroxide combination as described above (i.e., multiple injection), the organic peroxide according to the invention may be injected at one point of the reactor and the additional organic peroxide or peroxides as described above may be injected at one or other points of the reactor. In this case, the organic peroxide according to the invention and the additional organic peroxide or peroxides as described above are not formulated in the same composition, but belong to the same peroxidic initiator system, so as to ensure the entirety of the polymerization or copolymerization of the ethylene.

When a tubular reactor is used, the mixture of ethylene and optional comonomer(s) is preferably introduced at the top of the tubular reactor. The organic peroxide according to the invention or the combination of organic peroxides is injected by means of a high-pressure pump at the top of the reactor, after the location at which the mixture of ethylene and the comonomer(s) is introduced.

The mixture of ethylene and the optional comonomer(s) may be injected at at least one other point of the reactor, and this injection is in turn followed by another injection of the organic peroxide according to the invention or of a combination of organic peroxides as defined above, this being then referred to as a multipoint injection technique. When the multipoint injection technique is used, the combination is preferably injected in such a way that the weight ratio of the combination injected at the reactor inlet to the entirety of the combination injected is between 10% and 90%.

Other processes for high-pressure tubular polymerization or copolymerization which can be used are for example those described in US2006/0149004 A1 or in US2007/0032614 A1.

It is also possible to use an autoclave reactor for implementing the high-pressure radical polymerization or copolymerization of ethylene and the optional comonomers. An autoclave reactor generally consists of a cylindrical reactor into which a stirrer is placed. The reactor can be separated into several zones connected to one another in series.

The process according to the invention is preferably implemented in an autoclave reactor, especially in a multizone reactor.

Advantageously, the residence time in the reactor is between 30 and 120 seconds.

The organic peroxide according to the invention, alone or in combination with one or more additional organic peroxides, is also injected into this first reaction zone when the reaction zone reaches a temperature of between 150° C. and 200° C.

In the course of the reaction, the temperature may be between 150° C. and 320° C., since the reaction is exothermic. If the reactor is a multizone reactor, the stream of ethylene and of optional comonomers which have not reacted and also the polymer formed then pass through the subsequent reaction zones.

In each reaction zone, ethylene, optional comonomers and initiators can be injected, at an initiation temperature of between 160 and 190° C. The temperature of the zones after initiation is between 150 and 320° C.

The pressure of the reactor ranges from 500 bar (50 MPa) to 3000 bar (300 MPa), preferably from 1200 bar (120 MPa) to 3000 bar (300 MPa).

The aim of the process according to the invention is to prepare polyethylene or an ethylene-based copolymer.

The ethylene copolymer is preferably selected from the group consisting of copolymers of ethylene and acrylate(s), ethylene-vinyl acetate (EVA) copolymers, copolymers based on ethylene and one or more alpha- or alpha, omega-olefin monomers, copolymers based on ethylene and carbon monoxides, and copolymers based on ethylene and unsaturated cyclic anhydride comonomers.

The copolymer of ethylene and acrylate comprises at least one unit obtained from ethylene and at least one unit obtained from an acrylate.

The acrylate is especially selected from the group consisting of alkyl (meth)acrylates, more particularly C₁-C₃₀ alkyl (meth)acrylates, and arylalkyl (meth)acrylates, alkanol (meth)acrylates, such as hydroxyethyl (meth)acrylate, and (meth)acrylates containing an epoxy group.

The alkyl and arylalkyl groups may be linear or branched and may contain from 1 to 30 carbon atoms, preferably from 1 to 24 carbon atoms.

The alkyl and arylalkyl groups may also contain ether or thioether functions.

The alkyl (meth)acrylates are preferably selected from the group consisting of methyl acrylate, ethyl acrylate, n-butyl acrylate, isobutyl acrylate, 2-ethylhexyl acrylate, cyclohexyl acrylate, n-octyl acrylate, methyl methacrylate, ethyl methacrylate and butyl methacrylate, and mixtures thereof.

The (meth)acrylates containing an epoxy group are preferably selected from the group consisting of glycidyl methacrylate, glycidyl acrylate and mixtures thereof.

The acrylate is advantageously selected from the group consisting of alkyl (meth)acrylates, more particularly C₁-C₃₀ alkyl (meth)acrylates, and more particularly still C₁-C₂₄ alkyl (meth)acrylates.

The acrylate is selected more advantageously from the group consisting of methyl acrylate, butyl acrylate, 2-ethylhexyl acrylate, or mixtures thereof, especially butyl acrylate.

The aim of the process according to the invention is preferably to prepare low-density polyethylene (LDPE) or a copolymer of ethylene and vinyl acetate (EVA).

The aim of the process according to the invention more preferably still is to prepare polyethylene, preferably low-density polyethylene (LDPE).

The invention also relates to the ethylene polymer or copolymer obtained by the process above.

Composition

The present invention also relates to a composition comprising:

(i) at least one ethylene monomer,

(ii) at least one peroxide selected from the group consisting of hemiperoxyacetals, preferably having a half-life temperature at one minute and at atmospheric pressure of from 125° C. to 160° C., as described above, and

(iii) optionally at least one additional organic peroxide, distinct from the peroxide (ii), as described above.

The composition may further comprise one or more comonomers amenable to copolymerization with ethylene by a radical route under high pressure.

The comonomers are preferably selected from the group consisting of esters of unsaturated carboxylic acids (or salts thereof), anhydrides of carboxylic acids, vinyl esters, such as vinyl acetate or pivalate acetate, alpha-olefins such as propene, 1-butene, 1-hexene, 1-octene and 4-methyl-1-pentene, unsaturated carboxylic acids such as (meth)acrylic acid, maleic acid and fumaric acid, derivatives of (meth)acrylic acids such as (meth)acrylonitrile and (meth)acrylamide, vinyl ethers such as vinyl methyl ether and vinyl phenyl ether, and vinyl aromatic compounds such as styrene and alpha-methyl styrene, or mixtures thereof.

The comonomers or comonomers are more preferably selected from esters of unsaturated carboxylic acids (or salts thereof), vinyl esters, and mixtures thereof.

The esters of unsaturated carboxylic acids are preferably selected from the group consisting of alkyl (meth)acrylates, more particularly C₁-C₂₄ alkyl (meth)acrylates, and (meth)acrylates containing an epoxy group.

The alkyl (meth)acrylates are preferably selected from methyl acrylate, ethyl acrylate, n-butyl acrylate, isobutyl acrylate, 2-ethylhexyl acrylate, cyclohexyl acrylate, n-octyl acrylate, methyl methacrylate, ethyl methacrylate and n-butyl methacrylate.

The comonomer or comonomers are more preferably selected from the group consisting of vinyl ethers, especially vinyl acetate.

The composition according to the invention enables access to a polyethylene or an ethylene-based copolymer.

Preferably, the composition according to the invention comprises:
at least one ethylene monomer,
one or more organic peroxides selected from the group consisting of hemiperoxyacetals as defined above,
one or more additional organic peroxides selected from the group consisting of peroxyacetals as defined above.

The composition preferably also comprises one or more additional organic peroxides selected:
from the group consisting of peroxides having a half-life temperature at one minute and at atmospheric pressure which is less than that of the hemiperoxyacetal as defined above, or, in the case of several hemiperoxyacetals as defined above, of the hemiperoxyacetal having the lowest half-life temperature at one minute and at atmospheric pressure among the hemiperoxyacetals,
from the group consisting of peroxides having a half-life temperature at one minute and at atmospheric pressure which is more than the half-life temperature of the hemiperoxyacetal as defined above, or, in the case of several hemiperoxyacetals as defined above, of the hemiperoxyacetal having the highest half-life temperature at one minute and at atmospheric pressure among the hemiperoxyacetals,
from the group consisting of peroxy esters capable of initiating the radical polymerization or copolymerization of ethylene under high pressure at a temperature of from 160° C. to 190° C.,
from the group consisting of organic peroxides capable of initiating the radical polymerization or copolymerization of ethylene under high pressure at a temperature higher than 220° C.,
and mixtures thereof.
“At least one ethylene monomer” means that the composition comprises at least ethylene monomers, and optionally other comonomers as defined above.

The examples that follow serve to illustrate the invention without, however, being limiting in nature.

### EXAMPLES

### Example 1

Organic Peroxide Tested

In the example that follows, a radical polymerization of ethylene under high pressure was carried out, on the one hand with tert-butyl peroxy-2-ethylhexanoate (Luperox® 26), called the reference peroxide, and on the other hand with 1-methoxy-1-tert-amylperoxycyclohexane, TAPMC, called peroxide (1).

Peroxide (1) and the reference peroxide are two organic peroxides which are capable of initiating the radical reaction of ethylene under high pressure within a temperature range, referred to as medium temperature range, of from 160° C. to 190° C.

In this comparative example, the reference peroxide was replaced weight for weight by the peroxide (1).

The reference peroxide has a self-accelerating exothermic decomposition temperature (SADT) of 35° C., which means that it must be stored in a cold environment, at a temperature in the region of 5-10° C., and that particular precautions must be taken when transporting it.

Peroxide (1), for its part, has a self-accelerating exothermic deposition temperature (SADT) of 60° C., allowing it to be stored and transported at ambient temperature.

Experimental Protocol

In a 435 ml high-pressure stirred batch reactor of autoclave type, the ethylene is injected until a pressure of 1800 bar is reached, i.e. approximately 207 g. Stirring is at 1000 rpm (revolutions per minute). The initial temperature was established at the reactor wall temperature at 160° C. by means of heater rods placed in the walls of the reactor.

The peroxides (peroxide (1) and reference peroxide) are respectively diluted in heptane before being injected into the reactor.

A transfer agent, propionaldehyde, is also used in order to limit the molecular masses and the fouling of the reactor.

Thus each organic peroxide (peroxide (1) and the reference peroxide) is diluted in heptane, and the propionaldehyde upstream of the reactor and at low temperature, so as not to initiate the reaction prior to entry into the reactor. Each mixture is then injected into the reactor using a high-pressure pump. The polymerization is triggered as soon as the peroxide is injected at an initial temperature of 160° C.

During the radical polymerization reaction, the thermal evolution curve, which follows the introduction of each peroxide into the reactor and corresponds to the exotherm of polymerization of ethylene, is ascertained. The exothermic curve corresponds to the kinetics of the radical reaction.

The exothermic curve passes through a temperature maximum, referred to as the maximum temperature attained and recorded as Tmax.

Determinations are made of Tmax and also the rate at which this Tmax is attained for a given level of addition.

The reaction proceeds until the final temperature returns to the same level of value as the initial temperature.

The reactor is then depressurized and the resin is recovered for a measurement of the specific peroxide consumption.

Results

Attainment of Tmax

Under the operating conditions referred to above (initial temperature=160° C. and pressure=1800 bar), the Tmax values observed for the two peroxides, the reference peroxide tert-butyl peroxy-2-ethylhexanoate (Luperox® 26) and the 1-methoxy-1-tert-amylperoxycyclohexane (TAPMC) peroxide, and for a respective concentration in ppm by weight relative to the ethylene monomer of 46 and 45 ppm by relative weight, are attained respectively in: 8.3 s and 11.7 s.

These Tmax values are attained according to virtually superimposed kinetic curves, and the slightly slower attainment of the Tmax with the TAPMC according to the invention is due to the fact that the Tmax attained with this peroxide is higher: 243° C. as against 214° C. for the reference, in spite of virtually the same level of addition by weight.

Specific Consumption

The specific consumption of the peroxide (1) and of the reference peroxide is also measured for two radical polymerizations using the same levels of addition and enabling the attainment of the maximum temperature Tmax for each of said peroxides, namely of 230° C. and of 205° C., still with an initial temperature of 160° C.

TABLE 1
Specific peroxide consumption

| Tmax 230° C. | g commercial | |
|---|---|---|
| | initiator/kg LDPE | |
| Luperox ® 26 | 1.096 | 0 |
| TAPMC | 0 | 0.281 |
| Specific | 1.096 | 0.281 |
| consumption | | |
| compared | | |
| Tmax 205° C. | kg commercial | |
| | initiator/mt LDPE | |
| Luperox ® 26 | 0.587 | 0 |
| TAPMC | 0 | 0.181 |
| Specific | 0.587 | 0.181 |
| consumption | | |
| compared | | |

According to this example, it is found that the production of LDPE when using a single initiator allows a gain in specific consumption of the order of 70% in terms of commercial peroxide (that is, undiluted peroxide) with peroxide (1) (TAPMC) relative to the reference peroxide (Luperox® 26) which is generally considered to be preferred in the state of the art.

### Example 2

Organic Peroxides Tested

In the example which follows, a radical polymerization of ethylene under high pressure was carried out, with two organic peroxide cocktails containing respectively one peroxide of the medium range of operational temperatures (peroxide (1) or reference peroxide from example 1) combined with a more reactive organic peroxide and another less reactive organic peroxide, as defined above.

The two radical polymerizations of ethylene under high pressure were therefore carried out with the following peroxidic initiator systems:
ternary cocktail 1: tert-butyl peroxypivalate (Luperox® 11) (as the peroxide more reactive than the reference peroxide), the reference peroxide (tert-butyl peroxy-2-ethylhexanoate—Luperox® 26) and tert-butyl peroxy-3,5,5-trimethylhexanoate (Luperox® 270) (as the peroxide less reactive than the reference peroxide),
ternary cocktail 2: tert-butyl peroxypivalate (Luperox® 11) (as the peroxide more reactive than the reference peroxide (1)), peroxide (1) (1-methoxy-1-tert-amylperoxycyclohexane—TAPMC) and tert-butyl peroxy-3,5,5-trimethylhexanoate (Luperox® 270) (as the peroxide less reactive than the reference peroxide).

In the ternary cocktail 2, the reference peroxide was replaced weight for weight by the peroxide (1).

The ratio by mass of the three peroxides (Lup11/Lup26/Lup 270 and Lup11/TAPMC/Lup270) in their commercial presentation for each of the two ternary cocktails was in all cases 2:1:1.

Only Luperox® 11 was used in a diluted commercial form for safety reasons, at 75% in the phlegmatizer isododecane, in the commercial presentation of Luperox 11M75 (Lup11M75), the other peroxides being available in undiluted form. The ratio by mass of the Lup11/Lup26/Lup270 system therefore expresses a weight ratio between undiluted peroxidic active substances of 1.5/1/1 respectively, corresponding to the 2:1:1 by weight mixture of Lup11M75/Lup26/Lup270.

The ternary cocktail 1 is called the reference cocktail.

Experimental Protocol

The two radical polymerizations were carried out in the same batch reactor as that of example 1.

However, the initial temperature of the ethylene charge at 1800 bar was established at the lower initial temperature of 145° C., by virtue of the ternary cocktail, in which the most reactive organic peroxide dictates operation at a lower initial temperature than in example 1.

As in example 1, the ternary reference cocktail containing TBO and the cocktail 2 containing peroxide (1) were tested at the same overall level of addition in terms of organic peroxides in a first phase, in order to judge the kinetics (examination of the reaction exotherm climb ramp and Tmax attained as a function of time).

Result

Attainment of Tmax

As in example 1, the kinetic curves exhibit a very similar exothermic ramp, attaining Tmax in 12.3 seconds for the ternary reference cocktail, but in 14.2 seconds for the ternary cocktail 2 employing TAPMC in place of the Luperox® 26, at substitution weight for weight.

Again, this difference in time for attainment of Tmax is due to the fact that with peroxide (1), the Tmax was 256° C. as against 249° C. for the ternary reference cocktail, in spite of a total level of addition of peroxides, expressed in pure form, of 91 ppm by weight versus 113 ppm by weight for the ternary reference.

Specific Consumption

The specific consumption of the overall amounts of peroxides involved in the two ternary cocktails was also measured, at two maximum temperatures Tmax, namely of 250° C. and of 240° C., with an initial temperature of 145° C. for each of the cocktails.

TABLE 2
Specific overall consumption of peroxides

| Cocktail of peroxides | Tmax | Peroxides injected | Specific consumption |
|---|---|---|---|
| | in ° C. | (total in ppm by weight) | in g peroxide/kg LDPE |
| Luperox ® 11/Luperox ® 26/Luperox ® 270 | 250 | 116 | 0.898 |
| Luperox ® 11/TAPMC/Luperox ® 270 | 250 | 83 | 0.627 |
| Luperox ® 11/Lup26/Luperox ® 270 | 240 | 87 | 0.734 |
| Luperox ® 11/TAPMC/Luperox ® 270 | 240 | 55 | 0.454 |

According to this example, it is found that the production of LDPE profits from a gain in overall specific consumption of the order of 30% in terms of pure peroxide (that is, undiluted peroxide) with peroxide (1) (TAPMC) relative to Luperox® 26, which is generally considered to be preferred by those skilled in the art, when the ternary cocktail is a mixture with a composition including a more reactive peroxide and a less reactive peroxide than peroxide (1), which replaces Luperox® 26 according to the invention, and in spite of the presence of peroxide (1) at less than 30% by mass in the cocktail.

### Example 3

In the example which follows, a radical polymerization of ethylene under high pressure was carried out, with two organic peroxide cocktails containing respectively one peroxide of the medium range of operational temperatures (peroxide (1) or reference peroxide from example 1) combined with a more reactive organic peroxide and another less reactive organic peroxide, as defined above.

The two radical polymerizations of ethylene under high pressure were therefore carried out with the following peroxidic initiator systems:
ternary cocktail 3: tert-butyl peroxypivalate (Luperox® 11) (as the peroxide more reactive than the reference peroxide), the reference peroxide (tert-butyl peroxy-2-ethylhexanoate—Luperox® 26) and tert-butyl peroxy-3,5,5-trimethylhexanoate (Luperox® 270) (as the peroxide less reactive than the reference peroxide),
ternary cocktail 4: tert-butyl peroxypivalate (Luperox® 11), peroxide (1) (1-methoxy-1-tert-amylperoxycyclohexane—TAPMC) and 2,2-di(tert-amylperoxy)butane (peroxide (2) belonging to the formula (II)—Luperox® 520) (as the peroxide less reactive than peroxide (1)).

In the two ternary cocktails, the reference peroxide (Luperox® 26) was replaced weight for weight by peroxide (1), and the tert-butyl peroxy-3,5,5-trimethylhexanoate (Luperox® 270) was replaced weight for weight by 2,2-di(tert-amylperoxy)butane which belongs to the formula (II) described above (called peroxide (2)).
The ratio by mass of the three peroxides (Luperox® 11/Luperox® 26/Luperox® 270 and Luperox® 11/TAPMC/Luperox® 520) for each of the two cocktails in their commercial form is 2:1:1.
Luperox® 11 was used in a diluted commercial form for safety reasons, at 75% in the phlegmatizer isododecane, in the commercial presentation of Luperox® 11M75.
Luperox® 520 was used in a diluted form for safety reasons, at 50% in the phlegmatizer isododecane (Luperox® 520M50).
The ratio by mass of pure peroxides in the ternary cocktail 3 is 1.5/1/1 respectively in the order described above, and the ratio by mass of pure peroxides in the ternary cocktail 4 is 1.5/1/0.5.
The ternary cocktail 3 corresponds to a conventional peroxidic initiator system based on peroxy esters, and corresponds hereinafter to the reference cocktail.

Experimental Protocol

The two radical polymerizations were carried out in the same batch reactor as that of example 2 and under the same conditions, meaning that the initial temperature of the ethylene charge at 1800 bar was established at an initial temperature of 145° C.

As in example 1, the exothermic curve of the radical reaction, the maximum temperature attained and the specific consumption of peroxides are ascertained for each polymerization reaction. The characteristics of polymerization of ethylene are therefore compared for each ternary cocktail.

Results

The kinetic curves ascertained for each cocktail exhibit very similar exothermic ramps, attaining Tmax in 12.3 seconds for the ternary reference cocktail (ternary cocktail 3), and 11.7 seconds for the ternary cocktail 4.

However, in spite of a very much lower overall amount of peroxides engaged in the radical polymerization for the ternary cocktail 4 (48 ppm expressed as pure peroxides), relative to 113 ppm for the ternary cocktail 3, the maximum temperature (Tmax) attained with the cocktail 4 is 256° C., as against 249° C. for the reference cocktail.

This difference indicates a greater production of resin (polymer) with cocktail 4 according to the invention, with a smaller overall amount of peroxides engaged.

### Example 4

In this example, the experiment described in example 3 was repeated a number of times, retaining the same initial conditions (initial temperature=145° C. and pressure of 1800 bar) with the reference cocktail (ternary cocktail 3) and cocktail 4, by varying the total concentration of peroxides, still in the ratios indicated above (weight ratio of the commercial organic peroxides 2/1/1).

Result

The maximum temperature Tmax is greater in all cases when the overall concentration of peroxides increases within the cocktails tested. However, the ternary cocktail 4, comprising the peroxide (1)/peroxide (2) pairing, results in a higher Tmax, and therefore in a greater production of polymer, relative to the reference cocktail 3. The difference observed is of the order of 20° C., when the same overall amount by weight of peroxides is used, over an overall peroxide concentration range of from 20 to 130 ppm by weight, expressed in their commercial formulation.

The overall specific consumption for the ternary cocktail 4 is lower than that of the ternary cocktail 3, by at least 35%, for the same overall concentration of peroxides engaged.

### Example 5

The specific consumption of the overall amounts of peroxide involved in the two ternary cocktails described in example 3 was also measured at two maximum temperatures Tmax, namely of 240° C. and of 250° C., with an initial temperature of 145° C.

The total amounts of peroxides in the table below are expressed in ppm by weight of peroxides in their commercial dilution (at 75% in isododecane for Luperox® 11, called Lup11M75, at 50% in isododecane for Luperox® 520, then called Luperox® 520M50, Luperox® 270 and Luperox® 26 are not diluted and are therefore taken at 100% for their contribution in the ternary mixture).

TABLE 3
Overall specific consumption of peroxides

| Tmax = 240° C. | Commercial | Specific consumption | Conversion |
|---|---|---|---|
| | peroxides injected | in g of total | of ethylene |
| | (total in ppm by | commercial | in total % |
| | weight) | peroxides/kg LDPE | |
| [symbol] | [symbol] | [symbol] | [symbol] |
| [symbol] | by weight) | /kg LDPE | [symbol] |
| Luperox ® 11M75/Luperox ® 26/Luperox ® 270 | 86.6 | 0.734 | 10.4 |
| Luperox ® 11 M75/TAPMC/Luperox ® 520M50 | 45.9 | 0.323 | 13.9 |
| Gain | 47% | 56% | 33.6% |

[symbol]

indicates data missing or illegible when filed

TABLE 4
Overall specific consumption of peroxides

| Tmax = 250° C. | Commercial | Specific consumption | Conversion of |
|---|---|---|---|
| | peroxides injected | in g of | ethylene in % |
| | (total in ppm | total commercial | |
| | by weight) | peroxides/kg LDPE | |
| Luperox ® 11M75/Luperox ® 26/Luperox ® 270 | 116.2 | 0.898 | 11.5 |
| Luperox ® 11M75/TAPMC/Luperox ® 520M50 | 62.5 | 0.420 | 14.8 |
| Gain | 46% | 53% | 28.7% |

According to this example, it is found that for the same maximum temperature Tmax attained in a given zone of a reactor, the ternary cocktail according to the invention demonstrates better radical initiation performance, not only in terms of a reduction in specific consumption of the order of 50% but also in terms of conversion. Indeed, the commercial amount of peroxides enabling the maximum temperatures to be attained is lower by at least 45% with the ternary cocktail according to the invention.

1-21. (canceled)

22. An organic peroxide, for use alone or in combination with one or more distinct additional organic peroxides, selected from the group consisting of hemiperoxyacetals for the radical polymerization or copolymerization of ethylene under high pressure.

23. The organic peroxide of claim 22, which is selected from the group consisting of hemiperoxyacetals having a half-life temperature at one minute and at atmospheric pressure of from 125° C. to 160° C.

24. The organic peroxide of claim 22, which is selected from the group consisting of hemiperoxyacetals conforming to the general formula (I) below: 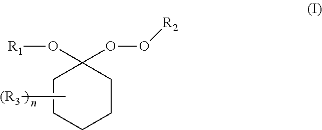
wherein in formula (I):
R₁ represents a linear or branched C₁-C₄,
R₂ represents a branched C₄-C₁₂,
n denotes zero or an integer from 1 to 3, and
R₃ represents a linear or branched C₁-C₃ alkyl group.

25. The organic peroxide of claim 22, wherein the organic peroxide is selected from the group consisting of 1-methoxy-1-tert-amylperoxycyclohexane (TAPMC), 1-methoxy-1-t-butylperoxycyclohexane (TBPMC), 1-methoxy-1-t-amylperoxy-3,3,5-trimethylcyclohexane, 1-methoxy-1-t-butylperoxy-3,3,5-trimethylcyclohexane, 1-ethoxy-1-t-amylperoxycyclohexane, 1-ethoxy-1-t-butylperoxycyclohexane, 1-ethoxy-1-t-butyl-3,3,5-peroxycyclohexane and mixtures thereof.

26. The organic peroxide of claim 22, wherein the organic peroxide is 1-methoxy-1-tert-amylperoxycyclohexane.

27. The organic peroxide of claim 22, wherein the additional peroxide or peroxides is or are selected from the group consisting of peroxyacetals.

28. The organic peroxide of claim 22, wherein the additional peroxide or peroxides is or are selected from the group consisting of peroxyacetals capable of initiating the radical polymerization or copolymerization of ethylene under high pressure in a temperature range of from 190° C. to 250° C.

29. The organic peroxide of claim 22, wherein the additional peroxide or peroxides is or are selected from the group consisting of peroxyacetals conforming to the general formula (II) below: 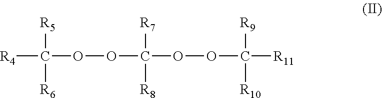
in which formula (II) R₄ to R₁₁, which are identical or different, represent a linear or branched C₁-C₆ alkyl group.

30. The organic peroxide of claim 22, wherein the additional peroxide or peroxides is or are selected from the group consisting of 2,2-di(tert-amylperoxy)propane, 2,2-di(tert-amylperoxy)butane and mixtures thereof.

31. The organic peroxide of claim 22, wherein the additional peroxide or peroxides are selected from the group consisting of peroxides having a half-life temperature at one minute and at atmospheric pressure which is lower than the half-life temperature of the hemiperoxyacetal as defined in claim 22.

32. The organic peroxide of claim 31, wherein the additional peroxide or peroxides is or are selected from the group consisting of tert-amyl peroxyneodecanoate, tert-butyl peroxyneodecanoate, diethylhexyl peroxydicarbonate, tert-amyl perpivalate, tert-butyl perpivalate, di(3,5,5-trimethylhexanoyl) peroxide, 2,5-dimethyl-2,5-di(2-ethylhexanoylperoxy)hexane, tert-amyl peroxy-2-ethylhexanoate and mixtures thereof.

33. The organic peroxide of claim 22, wherein the additional peroxide or peroxides are selected from the group consisting of peroxides having a half-life temperature at one minute and at atmospheric pressure which is higher than the half-life temperature of the hemiperoxyacetal as defined in claim 22.

34. The organic peroxide of claim 33, wherein the additional peroxide or peroxides is or are selected from the group consisting of tert-amyl peroxy-3,5,5-trimethylehexanoate, tert-butyl peroxy-3,5,5-trimethylhexanoate, tert-butyl peracetate, 2,2-di(tert-amylperoxy)butane, 2,2-di(tert-butylperoxy)butane, tert-amyl peroxybenzoate, tert-butyl peroxybenzoate and mixtures thereof.

35. The organic peroxide of claim 22, wherein the additional peroxide or peroxides are selected from the group consisting of peroxy esters which are capable of initiating the radical polymerization or copolymerization of ethylene under high pressure at a temperature of from 160° C. to 190° C.

36. The organic peroxide of claim 22, wherein the additional peroxide or peroxides are selected from the group consisting of peroxy esters which are capable of initiating the radical polymerization or copolymerization of ethylene under high pressure at a temperature higher than 220° C.

37. The organic peroxide of claim 22, for the radical polymerization of ethylene under high pressure.

38. A process for preparing polyethylene or an ethylene copolymer, comprising a step of radical polymerization or copolymerization of ethylene under high pressure, in the presence of at least one peroxide as defined in claim 22, alone or in combination with one or more distinct additional peroxides as defined in claim 22.

39. The process as claimed in claim 38, wherein the polymerization or copolymerization step is implemented by injecting the peroxide as defined in claim 22, alone or in combination with one or more distinct additional peroxides as defined in claim 22, at one point or at a plurality of points of a reactor.

40. The process as claimed in claim 38, wherein the ethylene copolymer is selected from the group consisting of copolymers of ethylene and acrylate(s), copolymers based on ethylene and at least one alpha- or alpha, omega-olefin copolymers, copolymers based on ethylene and carbon monoxides, and copolymers based on ethylene and unsaturated cyclic anhydride comonomers.

41. The process as claimed in claim 38, wherein the polymer for preparation is polyethylene.

42. A composition comprising:
i. at least one ethylene monomer,
ii. at least one peroxide selected from the group consisting of hemiperoxyacetals as defined in claim 22, and
iii. optionally at least one additional peroxide, distinct from the peroxide (ii), as defined in claim 22.
